(19) United States
(12) Patent Application Publication
(10) Pub. No.: US 2020/0078554 A1
(43) Pub. Date: Mar. 12, 2020

(54) Trans-radial access endovascular catheter and method of use

(71) Applicant: Daniel Ezra Walzman, Bergenfield, NJ (US)

(72) Inventor: Daniel Ezra Walzman, Bergenfield, NJ (US)

(21) Appl. No.: 16/602,469

(22) Filed: Oct. 11, 2019

Related U.S. Application Data
(63) Continuation-in-part of application No. 16/290,923, filed on Mar. 3, 2019.
Application No. 16/290,923 is a continuation-in-part of application No. 15/932,775, filed on Apr. 23, 2018.
Application No. 15/932,775 is a continuation-in-part of application No. 15/250,693, filed on Aug. 29, 2016.
Application No. 15/250,693 is a continuation of application No. 15/158,341, filed on May 18, 2016, now abandoned.

(51) Int. Cl. A61M 25/00; A61M 25/10; A61F 2/954; A61F 2/856; A61B 5/00

(52) U.S. Cl. CPC A61M 25/0041; A61M 25/10; A61M 25/0052; A61F 2/954; A61F 2002/9528; A61B 5/6852; A61B 5/4836; A61M 2210/12; A61F 2/856

(57) ABSTRACT

Devices and methods for establishing trans-radial access for medical intervention are described. In particular, some embodiments are optimized for consistently safely achieving complete cerebral angiography via a single trans-radial access site. A system may include a catheter with at least two active steering sites. Some embodiments may include at least one balloon. The methods include using said steering mechanisms to help guide and support said catheter. In some embodiments additional use is made of at least one vascular arch to provide further support and prevent kickback and prolapse of said catheter and any additional devices passed therethrough.

### CROSS-REFERENCE(S)

This is a non-provisional, continuation-in-part (CIP) utility application claiming the benefit of priority to U.S. Non-Provisional application Ser. No. 16/290,923 filed Mar. 3, 2019 which is CIP priority to Ser. No. 15/932,775 filed Apr. 23, 2018 and Ser. No. 15/250,693 filed Aug. 29, 2016 and Ser. No. 15/158,341 filed May 18, 2016, the entire contents of which are incorporated by reference and further to the following U.S. nonprovisional patent appl. Ser. No. 16/501,806, filed Jun. 10, 2019 (10 Jun. 2019); Ser. No. 16/501,592, filed May 2, 2019 (2 May 2019); Ser. No. 16/214,130, filed Dec. 9, 2018 (9 Dec. 2018); Ser. No. 16/151,335, filed Oct. 3, 2018 (3 Oct. 2018); Ser. No. 16/125,691, filed Sep. 8, 2018 (8 Sep. 2018); Ser. No. 16/100,351, filed Aug. 10, 2018 (10 Aug. 2018); Ser. No. 16/024,038, filed Jun. 29, 2018 (29 Jun. 2018); Ser. No. 16/013,707, filed Jun. 20, 2018 (20 Jun. 2018); Ser. No. 16/013,491, filed Jun. 20, 2018 (20 Jun. 2018); Ser. No. 15/998,041, filed Jun. 18, 2018 (18 Jun. 2018); Ser. No. 15/932,911, filed May 18, 2018 (18 May 2018); Ser. No. 15/932,906, filed May 18, 2018 (18 May 2018); Ser. No. 15/932,110, filed Feb. 5, 2018 (5 Feb. 2018); Ser. No. 15/732,955, filed Jan. 16, 2018 (16 Jan. 2018); Ser. No. 15/732,953, filed Jan. 16, 2018 (16 Jan. 2018); Ser. No. 15/732,397, filed Nov. 6, 2017 (6 Nov. 2017); Ser. No. 15/732,130, filed Sep. 20, 2017 (20 Sep. 2017) and Ser. No. 16/575,302, filed Sep. 18, 2019 (18 Nov. 2019); and to U.S. Pat. No. 10,258,371 B2, issued Apr. 16, 2019 (16 Apr. 2019).

### FIELD OF THE INVENTION

The described invention relates generally to endovascular devices and medical methods of use. More particularly, as alternative for traditional catheterization, including devices and methods for establishing expanded trans-radial vascular access.

### BACKGROUND OF THE INVENTION

Access to patient blood vessels is necessary for a wide variety of medical diagnostic and therapeutic purposes. Of interest to the present invention, are alternatives for traditional catheterization. While a wide variety of variations exist, the basic technique relies on access via a long and tortious path. Craniofacial angiography in particular has traditionally been performed through a transfemoral route. However, cardiac catheterization was previously also done similarly, and more recently is done more often via a trans-radial approach. Studies have demonstrated that trans-radial vascular access has lower access-site major complication rates than transfemoral access. However, access to contralateral carotid and vertebral circulations, as well as sometimes ipsilateral carotid circulations, can be quite difficult to accomplish in many patients with current catheter technologies, utilizing a trans-radial access approach. Any time an endovascular access route is difficult, increased risk is created.

With the introduction of a greater number and variety of intravascular techniques, including angioplasty, atherectomy, endovascular aneurysm repair, thrombectomy, minimally invasive cardiac surgery, and the like, a need has arisen to provide addition access ports for various procedures. As the number and size of procedures increase, so does the risk of complications which place individual patients at risk, and which are costly to the healthcare system.

For these reasons, it would be desirable to provide improved devices and methods as alternatives for traditional catheterization. In particular, it would be desirable to provide vascular access techniques which would be less injurious to a patient.

The prior art discloses the use of trans-radial arterial catheterization. Trans-radial for medical purposes means through, by way of, or employing the radial artery. More specifically, trans-radial access is used to perform medical catheterization procedures and for therapeutic procedures.

More recently, trans-radial access for medical intervention has become increasingly popular. The most advantageous aspect is very low access-site major bleeding complications even with aggressive use of anticoagulation and antiplatelet therapies. Often during such procedures, patients are given high doses of blood thinners and platelet inhibiting medications.

Although the current invention is optimal for trans-radial cerebral angiography, it can be used for many other applications as well. One such nonlimiting application is the catheterization of a contralateral internal mammary artery, which is often an important vessel needed to be accessed to do a complete heart catheterization in patients with prior bypass surgery. The current device can be used via other access sites as well.

Thus, a need arises to ameliorate said difficulties. At least some of these objectives will be met by the inventions described hereinafter.

Radial artery access is known in the art. Normally, said access is achieved with a short, bevel 21-gauge needle, and typically, a 0.018-0.021 guide wire. This smaller needle system allows for better control and pulsatile blood flow can be seen immediately. It is suggested during a radial artery catheterization to use a smaller needle than one traditionally used during femoral catheterization, which may reduce difficulty when obtaining access.

There are a variety of sheaths available on the market that may be suitable for radial access. There are some characteristics, however, that may be desired in a radial sheath such as a tapered edge and hydrophilic coating. The tapered edge allows for smooth insertion of the sheath, and a hydrophilic coating on the sheath reduces the incidence of radial artery spasm during trans-radial coronary procedures.

Although a JL 4 and JR 4 catheter can be used for left and right coronary artery cannulation, there are catheters on the market by various vendors designed specifically for radial artery access. These catheters have the common characteristic of a primary and secondary curve. A radial-specific catheter enables angiography of both right and left coronaries with a clockwise and counterclockwise rotation of one catheter. Eliminating catheter exchange can result in less total procedure time as well as fluoroscopy time and less incidence of radial artery spasm.

Additionally, the prior art discloses a set of Walzman radial access catheters, which can make safe percutaneous access of either carotid artery feasible in the vast majority of patients, for example Ser. No. 16/501,591. Said catheters may also reduce access-site complications further.

Catheters described herein can occasionally herein be further modified with at least one additional lumen substantially in the wall of said catheter, that can exit the wall of said catheter via at least one perforation in the outer wall of said catheter, to provide irrigation proximal to the balloon when said balloon is inflated, so as to minimize formation of clot proximal to said balloon. Such clots can form when a balloon occludes a vessel and causes stasis of blood.

The disclosed device can also be used alone, or in combination with additional catheters passed therethrough. The additional catheters may often be passed more distally in the vasculature. Additionally, sometimes the current catheter can be used to obtain access to a particular target vessel, and may then be exchanged out, utilizing standard exchange techniques, for a different catheter which may not have the wires embedded in its wall. Said different catheter may sometimes have a greater inner diameter for a given outer diameter, because its wall can be thinner, thereby allowing more procedures to be performed through a given size access artery.

### SUMMARY OF THE INVENTION

The present invention provides improved devices and method for establishing trans-radial arterial access to a multitude of end vessels, via a single site of radial access. While access can be established to a variety of particular blood vessels, including both arteries and veins, such as the femoral artery, radial artery, and the like, the advantage of accessing the radial artery is that it is likely to result in less harm to a patient.

The methods and devices of the present invention comprise particular improvements over the techniques and devices disclosed in the prior art. The present invention reduces the risk of injuring the tissue patient by increasing procedures that can be safely and effectively performed via a single radial access site. Notwithstanding the particular advantages of trans-radial access, said techniques and devices can also be occasionally used to allow procedures via other access sites, including but not limited to the brachial artery, axillary artery, femoral vessels, and other vessels as well.

### BRIEF DESCRIPTION OF THE DRAWINGS

FIG. 1 illustrates the vessels proximal to the aorta.

FIG. 2 illustrates the present invention using the right radial approach to the right vertebral artery.

FIG. 3 illustrates the present invention using the right radial approach to the right internal mammary artery.

FIG. 4 illustrates the present invention using the right radial approach to the right carotid artery.

FIG. 5 illustrates the present invention using the right radial approach to the left common carotid artery.

FIG. 6 illustrates the present invention using the right radial approach to left subclavian artery using an alternate technique.

FIG. 7 illustrates the present invention using the right radial approach to the left common carotid artery while implementing an arch fulcrum support configuration.

FIG. 8 illustrates the present invention using the right radial approach to the left vertebral artery, employing the lesser curve of the arch of the aorta as a vascular fulcrum.

FIG. 9 illustrates the present invention using the right radial approach to the left internal mammary artery.

FIG. 10 illustrates the present invention using the right radial approach to the left carotid artery, using an alternate technique, employing the lesser curve of the arch of the aorta as a vascular fulcrum.

FIG. 11 which illustrates the same device as FIG. 2 with the additional of a partially inflated traditional balloon or a partially inflated hydrogel balloon.

FIG. 12 which illustrates the same device as FIG. 3 with the additional of a partially inflated traditional balloon or a partially inflated hydrogel balloon.

FIG. 13 which illustrates the same device as FIG. 4 with the additional of a partially inflated traditional balloon or a partially inflated hydrogel balloon,

FIG. 14 which illustrates the same device as FIG. 5 with the additional of a partially inflated traditional balloon or a partially inflated hydrogel balloon.

FIG. 15 which illustrates the same device as FIG. 6 with the additional of a partially inflated traditional balloon or a partially inflated hydrogel balloon.

FIG. 16 which illustrates the same device as FIG. 7 with the additional of a partially inflated traditional balloon or a partially inflated hydrogel balloon.

FIG. 17 which illustrates the same device as FIG. 8 with the additional of a partially inflated traditional balloon or a partially inflated hydrogel balloon.

FIG. 18 which illustrates the same device as FIG. 9 with the additional of a partially inflated traditional balloon or a partially inflated hydrogel balloon.

FIG. 19 which illustrates the same device as FIG. 10 with the additional of a partially inflated traditional balloon or a partially inflated hydrogel balloon.

### DETAILED DESCRIPTION OF THE INVENTION

The present invention can be used for many procedures, but optimal for 4 and 6 vessel cerebral angiograms via a single radial artery approach, as well as complete heart catherization via a single radial approach in patients with internal mammary artery bypass on opposite side from radial access (i.e.—right radial access and left mammary artery or bilateral mammary artery bypass).

The present invention is introduced to a patient via an access needle (not shown), wherein said access needle penetrates a patient's radial artery. Most often the same needle first penetrates the skin above, and then is advanced into the radial artery, utilizing a percutaneous trans-radial approach. After penetration, an insertion wire functioning as a delivery rail is fed through said access needle. Said feed allows the rail wire to extend through said patient's vessels. Once said rail wire is established in a targeted vessel, the needle is removed and the wire is left in place. A primary catheter/sheath is then advanced over the rail wire. Subsequently, a secondary or working catheter is inserted into the primary catheter/sheath, and fed to a targeted area. Said secondary/working catheter is used as the access lumen for working wires, balloons, stents, and other medical devices. This set of elements is well known in the prior art, and not therefore shown in the figures. Occasionally, no sheath is used, and a secondary/working catheter is used alone, in an orientation sometimes referred to as “bareback”. This allows a larger working catheter to be used via a trans-radial approach. The device of the current invention can be used in any configuration.

Generally, the present invention discloses a device which has the following characteristics: a primary catheter having a length of approximately 10 cm-180 cm, and an external diameter 0.5Fr-35Fr, and incorporating at least one working lumen. The preferred embodiment has a single central working lumen spanning from a proximal end hole to a distal a distal end hole-said single working lumen capable of acting as a conduit for the delivery of additional catheters, wires, interventional devices, and other endovascular devices therethrough, which can optionally be advance more distally in the desired vasculature.

More specifically, the present invention discloses a device with a proximal end hole has an adjacent external termination device. It may also have a Luer lock and diaphragm. It also has at least two “pulley” wires to actively steer segments of the device. All steering wires are located substantially in the wall of the catheter.

The present invention discloses a device including a rotating wheel or pulley system or other activating mechanism, one per wire, to steer that segment— each wire steers only a single segment only in a single direction of wire pull. An optional embodiment has additional lumens substantially in its wall as well, which would serve exclusively for additional optional wires, or can serve exclusively as a conduit to inflate and deflate at least one optional balloon on an outer surface of the primary catheter. In such embodiment, the lumen used solely for inflation/deflation of said at least one balloon is disposed substantially within the wall of said device within the effective segment of the primary catheter. The term effective segment refers to that area or element of said primary catheter that traverses within the body of a patient. Said lumen used solely for inflation/deflation may branch away from the wall of the catheter along the proximal portion of said device outside the body.

Another embodiment has a single circumferential balloon near its distal end, which when inflated can alter flow in the vessel, and/or can serve as an anchor for the catheter. Some embodiments can have more than one balloon as well. Additional embodiments can have at least one irrigation lumen substantially within its wall along the intravascular segment as well. All lumens substantially within the wall of the catheter may exit said wall of said catheter and branch off at a point proximal to the skin access site.

The device disclosed by the present invention may use a balloon structure. It should be noted that any place a balloon structure is disclosed, said structure may be substituted by a hydrogel element. Optionally, the present invention may have hydrogel instead— which can act as a “balloon” by hydrating or substantially dehydrating in the presence of blood/fluid in response to an additional stimulus, and can thereby act to alter flow and/or as an anchor.

The present invention discloses a device with at least two steering segments: the primary steering segment— located along any desired segment from the distal end hole to a point 8 cm proximal to said end hole (i.e.—can be along last 1 cm of catheter in one version, can be from −2 cm to −4 cm in another), said primary segment being at least 0.1 cm long and no longer than 7 cm long. The primary steering segment is capable of being actively curved/bent from zero degrees to 180 degrees, said bends referring to an “after-bend” as defined further below.

The secondary steering segment is located proximal to said first steering segment, along any secondary segment of catheter between 2 cm and 30 cm from the distal end hole, wherein said secondary segment is at least 0.4 cm long and no more than 15 cm long.

Said secondary steering element is capable of steering and creating a bend/curve, via a pulley effect on its dedicated wire, of zero degrees to 270 degrees. Said secondary curve/bend is always on the same side and curving in substantially the same initial fashion as the primary curve/bend.

In an alternative embodiment, the present invention can have at least one additional pulley wire, to create at least one additional active steering segment(s) capable of creating at least one additional curve/bend (note— when pulley wires are oriented in different directions, steering segments may overlap along a length of the same catheter).

In the preferred embodiment there is a third/tertiary bend/curve, located along a segment at least 0.4 cm long, wherein said segment is located between zero cm and 8 cm from the distal end hole, and wherein said tertiary bend is in a substantially opposite direction from said primary and secondary bends. More specifically, said bends can be used through an external sheath, or “bare-back” with no external sheath. In some embodiments, such as those with an external balloon, a “peel-away” sheath can be used to insert the distal catheter segment (i.e.—portion w balloon), and the peel away sheath can then be removed and peeled away, and remainder of device can be used “bare-back” when desired. In some alternate versions a peel-away sheath can also be used as an introducer into an outer sheath as well.

In the preferred embodiment there is also a removable inner “dilator” which aids with insertion, and reduces the “shelf” between the current catheter and any insertion wire. Said inner dilator has an outer diameter that is less than the inner diameter of the current catheter (for any individual catheter), and a length greater that of the current catheter

In many cases, especially diagnostic angiograms, the current device can be the only catheter used. In other cases, including many but not all diagnostic angiograms and many but not all interventions, the current catheter may be used with at least one additional catheter that is passed through the current device. The second inner catheter, wire, and or other devices are most often passed into the more distal vasculature. Said current device and its curves, in such cases, aid in appropriate positioning and directional assistance for advancing such secondary inner structures. Said active pulley wires also provide support to prevent kickback and prolapse of the current device as well, as said additional inner devices advanced distally therethrough.

In many embodiments, said device of the current catheter is further optimized for support by utilizing a vascular arch, especially the lesser curve of the aortic arch, and resting a portion of the current catheter along a vascular arch, thereby providing further additional support, and further minimizing the occurrences of kickback and prolapse of the current device and/or any additional inner devices passed therethrough; as has been previously described in a prior patent by Walzman, specifically, U.S. patent application Ser. No. 16/290,923, filed 3 Mar. 2019; and U.S. Pat. No. 10,258,371, issued 16 Apr. 2019.

In many diagnostic and interventional procedures, the device disclosed in the present invention can act as the sole “guide” catheter for placing additional medical devices therethrough. Alternatively, in other interventional cases, an additional inner “guide” catheter can be passed through the current device, and more distally in the vasculature, in order to accommodate additional medical devices therethrough. In still other procedures the current device can be used, with or without additional inner wires and/or catheters, in order to optimally place an “exchange wire” and/or other similar exchange device into a target area— via the optimal steering and bending capabilities of the current device, and the current device can then be exchanged out for and replaced with a different catheter which will advance over said wire and/or other exchange device. Thereby in some cases a catheter with a larger Inner Diameter (ID) can be used (for a corresponding Outer Diameter-OD), allowing delivery of still more additional interventional devices for a given size of a patient's radial artery. Since the current invention has at least two wires substantially in its walls, catheters without said wires can be made with thinner walls and can thereby have a larger maximal ID for a given OD (ID— Inner diameter; OD— outer diameter). In various cases, various embodiments of the current invention with various segment lengths can be chosen, depending on the desired region(s) to access and on the patient's particular anatomy.

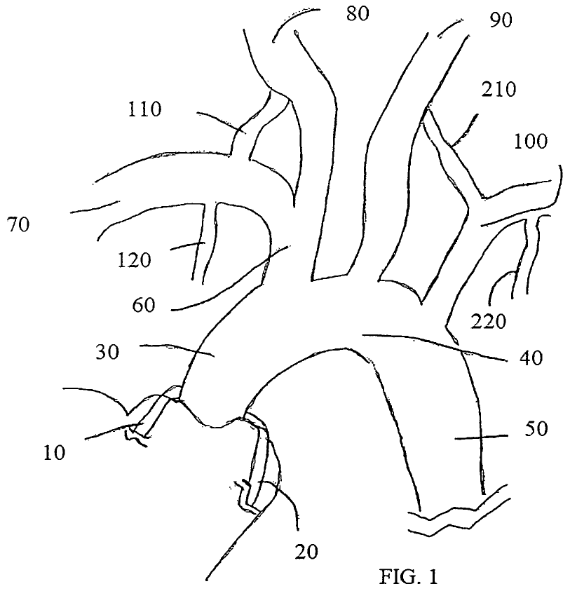

Now referring to FIG. 1 which illustrates the vessels proximal to the aorta, including the right coronary artery 10, the left coronary artery 20, ascending aorta 30, arch of aorta 40, descending aorta 50, brachiocephalic artery 60, right subclavian artery 70, right common carotid artery 80, left common carotid artery 90, and left subclavian artery 100. Additionally, illustrated the right vertebral artery 110, the right internal mammary artery 120, left vertebral artery 210 and left internal mammary artery 220.

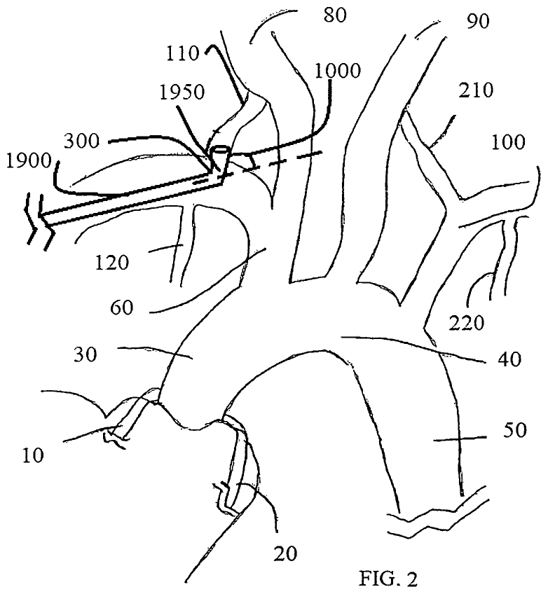

Now referring to FIG. 2 which illustrates the present invention using the right radial approach to the right vertebral artery 110. The after-bend angle 1000 is defined as the angle based on the direction of the catheter element 1900 before the catheter bend/curve 300 and the after-direction of the catheter element 1950 after the catheter bend/curve 300. The dashed lines indicate the direction of the catheter element 1900 before the catheter bend/curve 300.

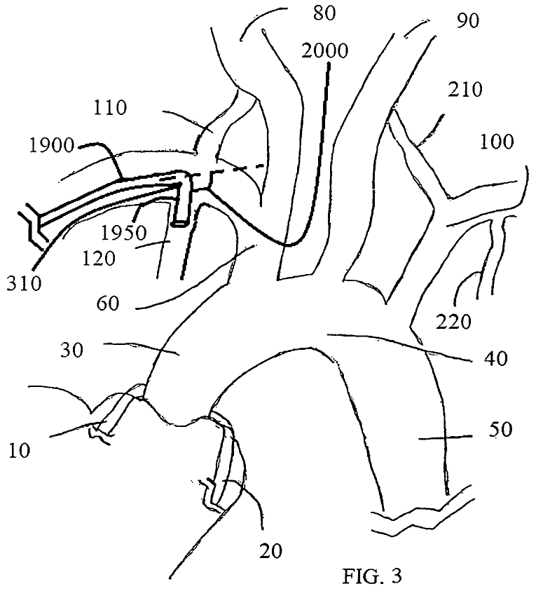

Now referring to FIG. 3 which illustrates the present invention using the right radial approach to the right internal mammary artery 120. The after-bend angle 2000 is defined as the angle based on the direction of the catheter element 1900 before the catheter bend/curve 310 and the after the direction of the catheter element 1950 after the catheter bend/curve 310. The dashed lines indicate the direction of the catheter element 1900 before the catheter bend/curve 3000. Catheter bend/curve 310 is also known as the tertiary bend. Alternatively, it may represent the primary bend of the present invention when the catheter of the present invention is rotated.

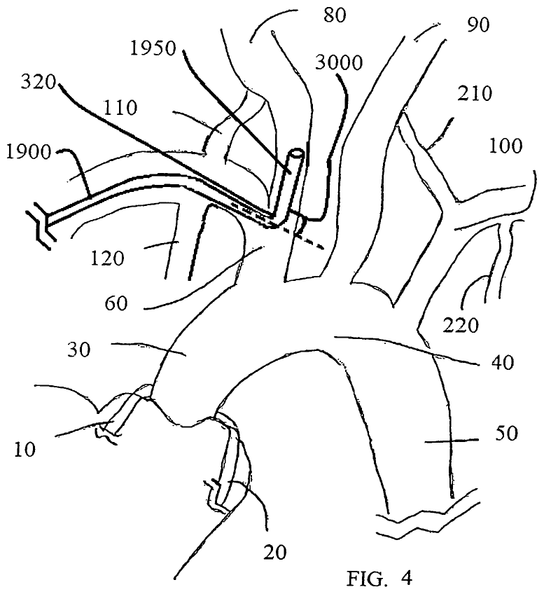

Now referring to FIG. 4 which illustrates the present invention using the right radial approach to the right common carotid artery 80. The after-bend angle 3000 is defined as the angle based on the direction of the catheter element 1900 before the catheter bend/curve 320 and the after the direction of the catheter element 1950 after the catheter bend/curve 320. The dashed lines indicate the direction of the catheter element 1900 before the catheter bend/curve 320. The primary outer curve/bend is the after-bend angle 3000 for this embodiment of the present invention.

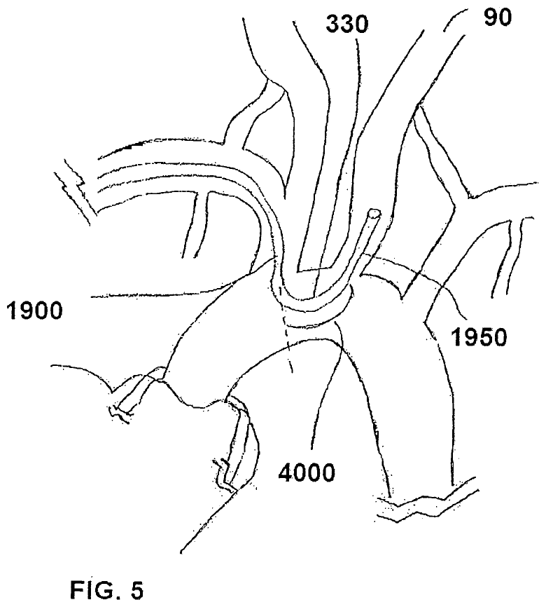

Now referring to FIG. 5 which illustrates the present invention using the right radial approach to the left common carotid artery 90. The after-bend angle 4000 is defined as the angle based on the direction of the catheter element 1900 before the catheter bend/curve 330 and the after the direction of the catheter element 1950 after the catheter bend/curve 330. The dashed lines indicate the direction of the catheter element 1900 before the catheter bend/curve 330.

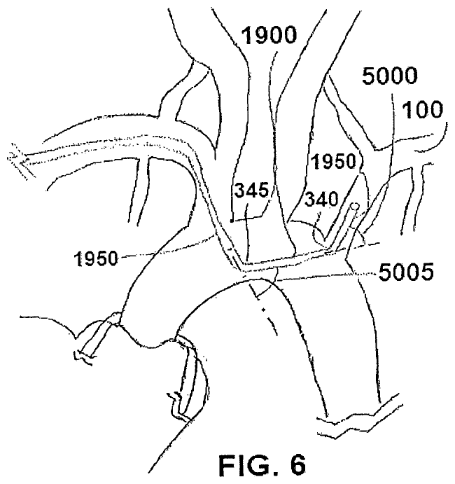

Now referring to FIG. 6 which illustrates the present invention using the right radial approach to the left subclavian artery 100. The primary after bend angle 5000 is defined as the angle based on the direction of the catheter element 1900 before the catheter bend/curve 340 and the after the direction of the catheter element 1950 after the catheter bend/curve 340 the curve 5000. The dashed lines indicate the direction of the catheter element 1900 before the catheter bend/curve 340. The secondary after bend angle 5005 is defined as the angle based on the direction of the secondary second catheter element 1905 before the catheter bend/curve 345 and the after the direction of the second catheter element 1900 after the catheter secondary bend/curve 345, marked as curve 5005. The combination of dashed and dotted lines indicates the direction of the secondary second catheter element 1905 before the catheter bend/curve 345. This illustrates another technique of using the present invention.

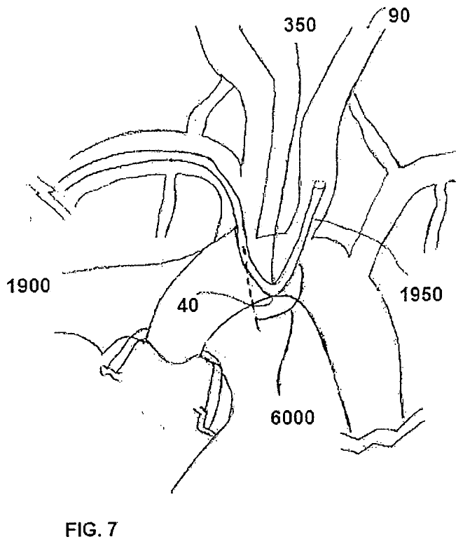

Now referring to FIG. 7 which illustrates the present invention using the right radial approach to the left common carotid artery 90 which implementing an arch fulcrum support configuration. The after-bend angle 6000 is defined as the angle based on the direction of the catheter element 1900 before the catheter bend/curve 350 and the after the direction of the catheter element 1950 after the catheter bend/curve 350, marked as the curve 6000. This illustrates an alternate technique of using the present invention which has the device of the disclosed invention resting on the arch fulcrum 40, thereby using the Walzman arch fulcrum support technique, as described in U.S. Pat. No. 10,258,371, for example. The same devices can also be used via a left radial approach.

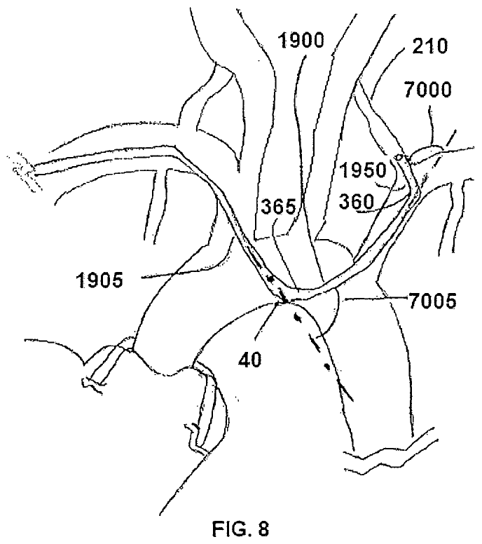

Now referring to FIG. 8 which illustrates the present invention using the right radial approach to the left vertebral artery 210 while implementing an arch fulcrum support configuration. The primary after bend angle 7000 is defined as the angle based on the direction of the catheter element 1900 before the catheter bend/curve 360 and the after the direction of the catheter element 1950 after the catheter bend/curve 360, marked as the curve 7000. The dashed lines indicate the direction of the catheter element 1900 before the catheter bend/curve 360. The secondary after bend angle 7005 is defined as the angle based on the direction of the secondary second catheter element 1905 before the catheter bend/curve 365 and the after direction of the second catheter element 1900 after the catheter bend/curve 365. The combination of dashed and dotted lines indicates the direction of the secondary second catheter element 1905 before the catheter bend/curve 365. This illustrates an alternate technique of using the present invention which has the present invention rest on the arch fulcrum 40 and thereby using the Walzman arch fulcrum support technique.

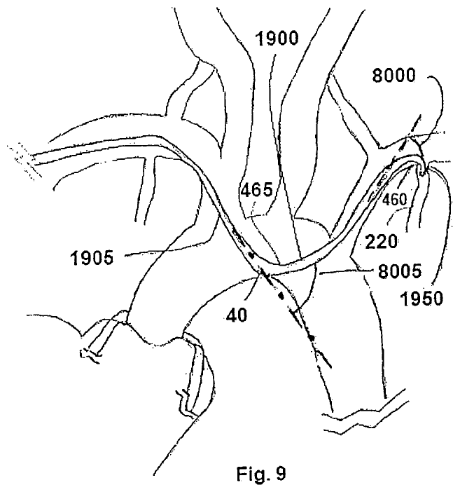

Now referring to FIG. 9 which illustrates the present invention using the right radial approach to the internal mammary artery 220. The primary after bend angle 8000 is defined as the angle based on the direction of the catheter element 1900 before the catheter bend/curve 460 and the after the direction of the catheter element 1950 after the catheter bend/curve 460. The dashed lines indicate the direction of the catheter element 1900 before the catheter bend/curve 460. The secondary after bend angle 8005 is defined as the angle based on the direction of the secondary second catheter element 1905 before the catheter bend/curve 465 and the after the direction of the second catheter element 1900 after the catheter bend/curve 465. The combination of dashed and dotted lines indicates the direction of the secondary second catheter element 1905 before the catheter bend/curve 465. This illustrates an optional technique of using the present invention which has the present invention rest on the arch fulcrum 40 and thereby using the Walzman arch fulcrum support technique.

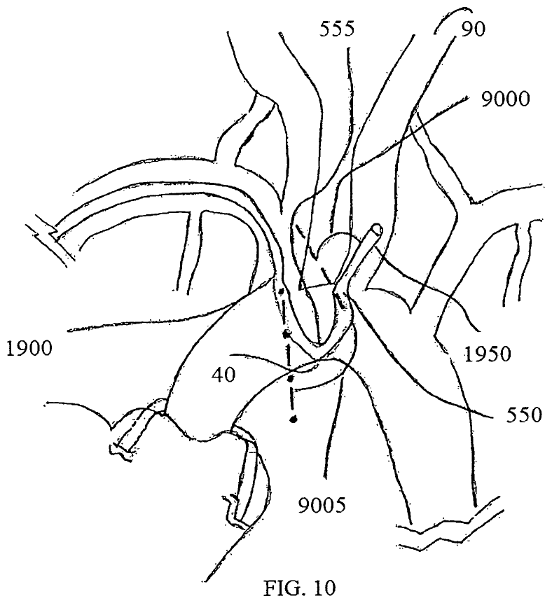

Now referring to FIG. 10 which illustrates the present invention using the right radial approach to the left common carotid artery 90, using an optional arch fulcrum technique. The primary after bend angle 9000 is defined as the angle based on the direction of the catheter element 1900 before the catheter bend/curve 550 and the after the curve direction of the catheter element 1950 after the catheter bend/curve 550. The dashed lines indicate the direction of the catheter element 1900 before the catheter bend/curve 550. The secondary after bend angle 9005 is defined as the angle based on the direction of the secondary second catheter element 1905 before the catheter bend/curve 555 and the after-direction of the second catheter element 1900 after the catheter bend/curve 555. The combination of dashed and dotted lines indicates the direction of the secondary second catheter element 1905 before the catheter bend/curve 555. This illustrates an optional preferred technique of using the present invention which has the present invention rest on the arch fulcrum 40 and thereby using the Walzman arch fulcrum support technique.

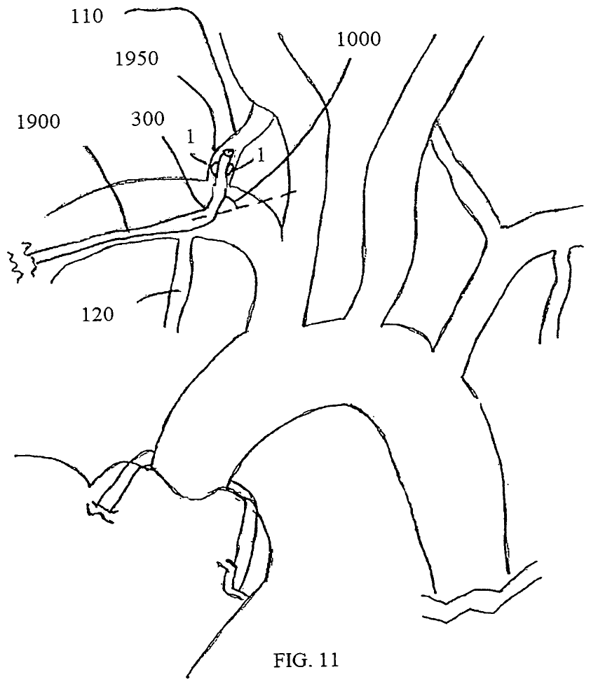

Now referring to FIG. 11 which illustrates the same device as FIG. 2 with the additional of a partially inflated traditional balloon or a partially inflated hydrogel balloon 1.

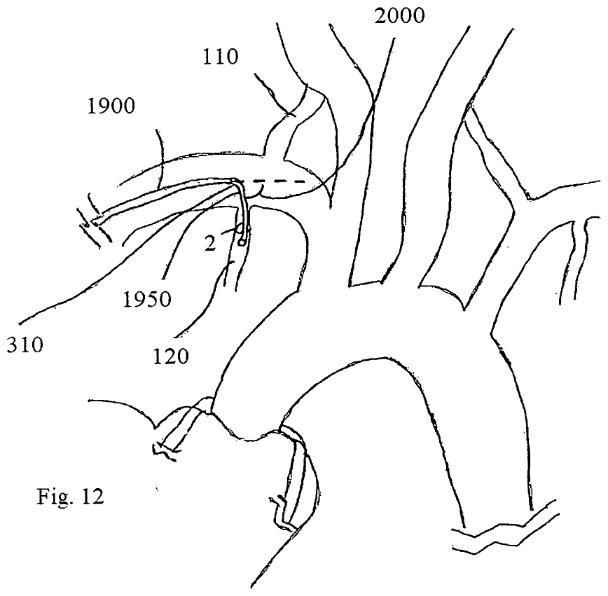

Now referring to FIG. 12 which illustrates the same device as FIG. 3 with the additional of a partially inflated traditional balloon or a partially inflated hydrogel balloon 2.

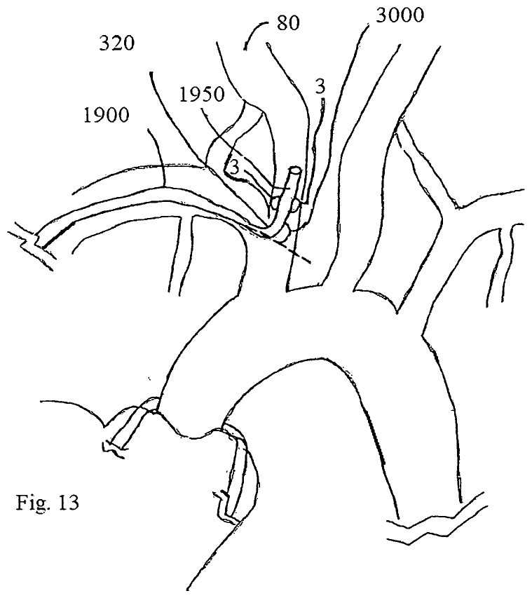

Now referring to FIG. 13 which illustrates the same device as FIG. 4 with the additional of a partially inflated traditional balloon or a partially inflated hydrogel balloon 3.

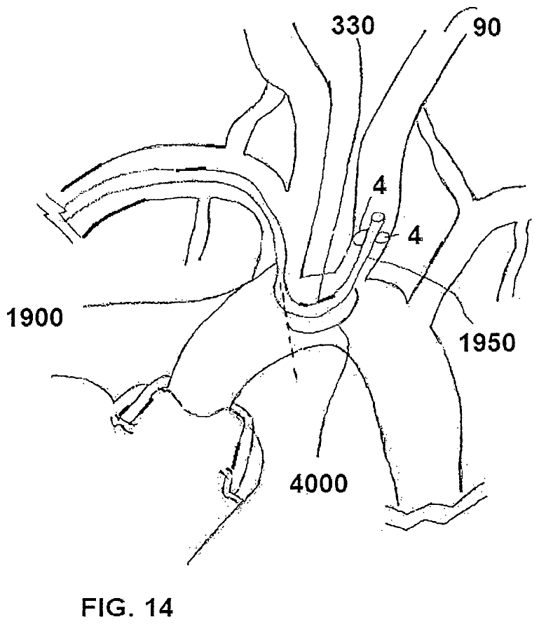

Now referring to FIG. 14 which illustrates the same device as FIG. 5 with the additional of a partially inflated traditional balloon or a partially inflated hydrogel balloon 4.

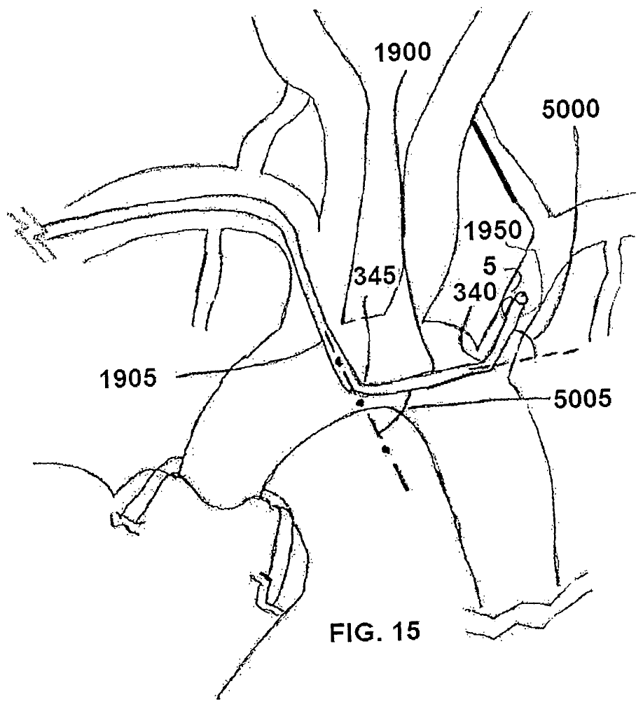

Now referring to FIG. 15 which illustrates the same device as FIG. 6 with the additional of a partially inflated traditional balloon or a partially inflated hydrogel balloon 5.

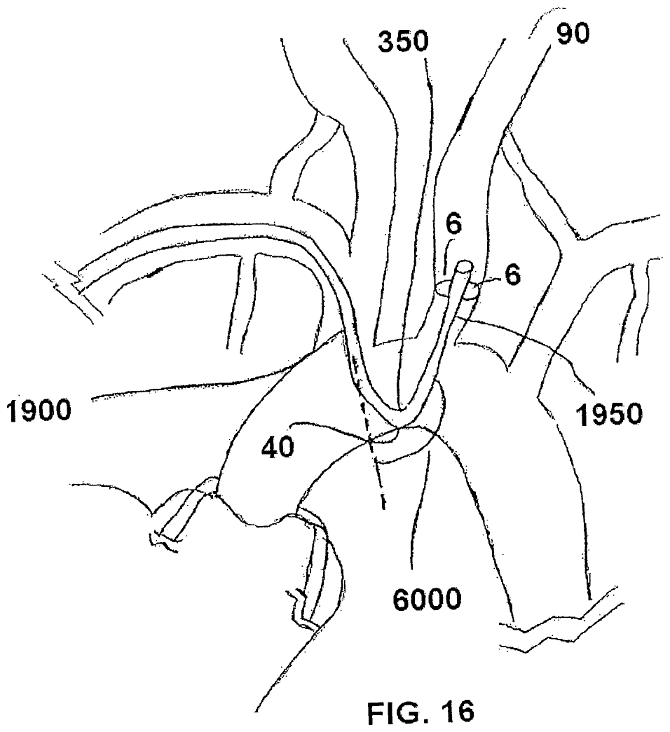

Now referring to FIG. 16 which illustrates the same device as FIG. 7 with the additional of a partially inflated traditional balloon or a partially inflated hydrogel balloon 6.

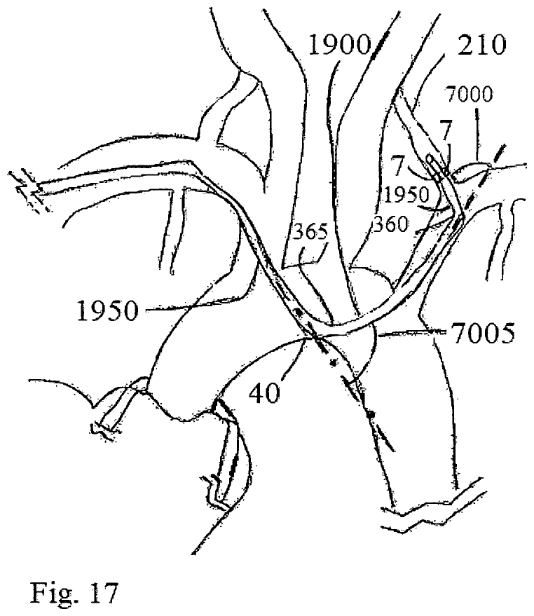

Now referring to FIG. 17 which illustrates the same device as FIG. 8 with the additional of a partially inflated traditional balloon or a partially inflated hydrogel balloon 7.

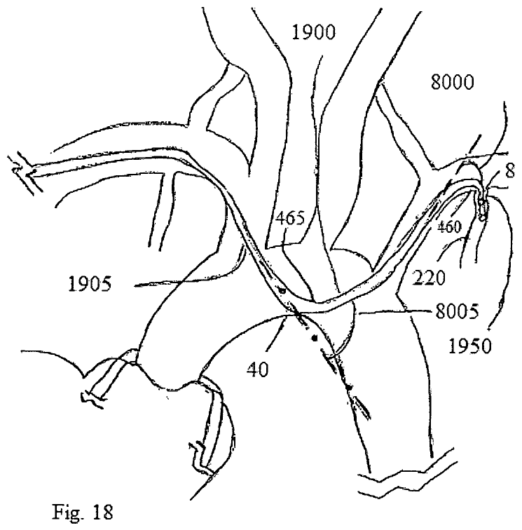

Now referring to FIG. 18 which illustrates the same device as FIG. 9 with the additional of a partially inflated traditional balloon or a partially inflated hydrogel balloon 8.

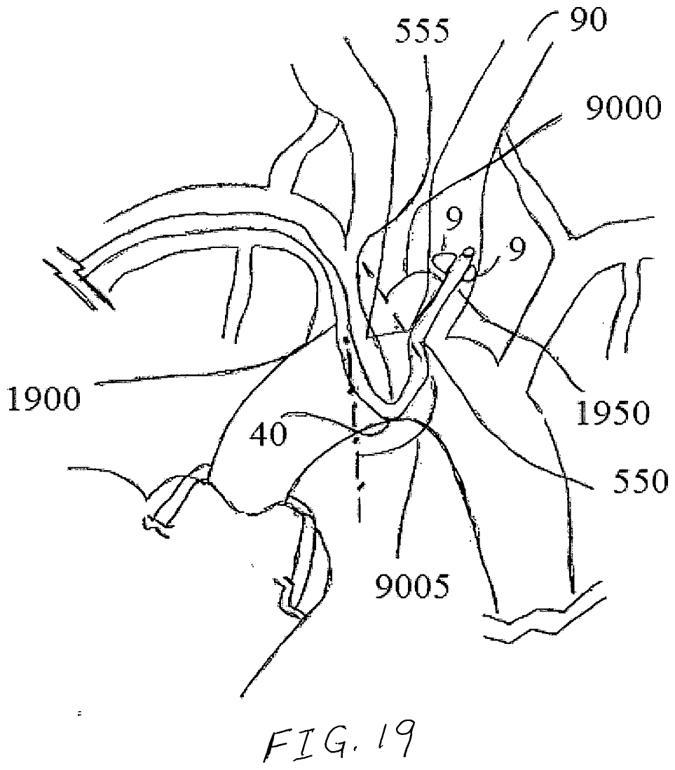

Now referring to FIG. 19 which illustrates the same device as FIG. 10 with the additional of a partially inflated traditional balloon or a partially inflated hydrogel balloon 9.

It should be noted that the prior art has variously defined calculations of bend angles from an inner curve or outer curve perspective. Additionally, prior art references are often ambiguous as to which measurement perspective is being described. Said angles described herein is a measured starting from a line drawn straight from the catheter before a bend/curve and extending straight beyond said bend/curve. This disclosure, therefore, includes sample bend angulation notations to optimally describe the angles referred to herein. Still further, the drawings associated with the disclosure of the present invention depict a right radial approach but left radial approach can also be used. Additional approaches and device uses are optionally possible as well. The devices are also optimally designed for percutaneous use. Notwithstanding this, uses via other approaches, including those that are not percutaneous, are possible as well.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

Any publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of e present invention. All such modifications are intended to be within the scope of the claims appended hereto.

What is claimed is:

1. A device for establishing trans-radial arterial access to a multitude of end vessels, via a single site of radial access comprising:
an access needle;
wherein said needle is capable of delivering a rail wire for delivery of a primary catheter;
said primary catheter having a single central working lumen, said single central working lumen having a proximal end hole, and a distal end hole;
said primary catheter having at least two steerable segments;
wherein said central working lumen is capable of acting as a conduit for the delivery therethrough of:
at least one additional secondary catheter,
at least one wire separate from said primary catheter, and
at least two pulley wires,
wherein said at least two pulley wires are located axially substantially within the wall of said single primary catheter, and capable of actively steering said at least two steerable segments; and
at least one pulley steering system for each of said at least two pulley wires;
wherein said at least two steerable segments each comprise said at least one pulley steering wire system for each segment, each of said at least one pulley steering system capable of steering each of said at least two steerable segments in a single direction of pulley wire pull,
said at least two steerable segments are composed of a primary steering segment and a secondary steering segment;
wherein said primary steering segment is:
located along any desired portion of said primary catheter within 10 cm of said distal end hole;
at least 0.1 cm long and no longer than 5 cm long; and
capable of being actively curved/bent from zero degrees to 180 degrees
wherein said secondary steering segment is:
located along any secondary segment located along a segment of said primary catheter, proximal to said primary segment, between 2 cm and 30 cm from said distal end hole;
at least 0.4 cm long and no more than 15 cm long,
capable of steering and creating a bend/curve, via a pulley effect on said pully wire, of zero degrees to 270 degrees; and
disposed on the same side, and curving in substantially the same initial fashion, as the primary curve/bend;
an external termination device which is in communication with said proximal end hole.

2. The device of claim 1, said primary catheter further comprising at least one balloon affixed to the outer surface and proximal to said distal end hole; and at least one additional lumen substantially within the wall of the effective segment of said primary catheter which functions to inflate and deflate said at least one balloon.

3. The device of claim 2, wherein said at least one balloon is capable of inflating and thereby are capable of:
altering flow in targeted vessels, and
anchoring said device.

4. The device of claim 3 further comprising a peel-away sheath.

5. The device of claim 1, additionally composed of hydrogel balloons affixed to the outer surface and proximal to said distal end holes;
wherein said hydrogel balloons are capable of swelling to effect inflation.

6. The device of claim 5, further comprising a peel-away sheath.

7. The device of claim 1, further comprising at least one additional tertiary steering segment and mechanism, located along a segment at least 0.4 cm long, wherein said segment is located between zero cm and 15 cm from the distal end hole, and wherein said tertiary bend is in a substantially different direction form said primary and secondary bends.

8. The device of claim 3, further comprising at least one additional tertiary steering segment and mechanism, located along a segment at least 0.4 cm long, wherein said segment is located between zero cm and 15 cm from the distal end hole, and wherein said tertiary bend is in a substantially different direction form said primary and secondary bends.

9. The device of claim 5, further comprising at least one additional tertiary steering segment and mechanism, located along a segment at least 0.4 cm long, wherein said segment is located between zero cm and 15 cm from the distal end hole, and wherein said tertiary bend is in a substantially different direction form said primary and secondary bends.

10. The device of claim 1, further comprising a removeable inner dilator to aid with percutaneous insertion, wherein the length of said inner dilator is greater than the length of the primary catheter and the outer diameter of the inner dilator is less than the inner diameter of the primary catheter.

11. The device of claim 1, further comprising at least one secondary catheter, capable of being advanced through said primary catheter.

12. The device of claim 1, further comprising at least one interventional treatment device.

13. The device of claim 1, further comprising at least one exchange device.

14. The device of claim 11, further comprising at least one interventional treatment device.

15. The device of claim 13, further comprising at least one interventional treatment device.

16. The device of claim 11, further comprising at least one exchange device.

17. The device of claim 16, further comprising at least one percutaneous treatment device.

18. The device of claim 7, further comprising at least one secondary catheter, capable of being advanced through said primary catheter.

19. The device of claim 7, further comprising at least one interventional treatment device.

20. The device of claim 7, further comprising at least one exchange device.

21. The device of claim 18, further comprising at least one interventional treatment device.

22. The device of claim 20, further comprising at least one interventional treatment device.

23. The device of claim 18, further comprising at least one exchange device.

24. The device of claim 23, further comprising at least one percutaneous treatment device.

25. The device of claim 8, further comprising at least one secondary catheter, capable of being advanced through said primary catheter.

26. The device of claim 8, further comprising at least one interventional treatment device.

27. The device of claim 8, further comprising at least one exchange device.

28. The device of claim 25, further comprising at least one interventional treatment device.

29. The device of claim 27, further comprising at least one interventional treatment device.

30. The device of claim 25, further comprising at least one exchange device.

31. The device of claim 31, further comprising at least one percutaneous treatment device.

32. The device of claim 3, further comprising at least one secondary catheter, capable of being advanced through said primary catheter.

33. The device of claim 3, further comprising at least one interventional treatment device.

34. The device of claim 3, further comprising at least one exchange device.

35. The device of claim 32, further comprising at least one interventional treatment device.

36. The device of claim 34, further comprising at least one interventional treatment device.

37. The device of claim 32, further comprising at least one exchange device.

38. The device of claim 34, further comprising at least one percutaneous treatment device.

39. The device of claim 34, further comprising at least one treatment device.

40. A device for establishing trans-radial arterial access to a multitude of end vessels, via a single site of radial access comprising:
an access needle;
wherein said needle is capable of delivering a rail wire for delivery of a primary catheter;
said primary catheter having a single central working lumen, said single central working lumen having a proximal end hole, and a distal end hole;
said primary catheter having at least two steerable segments;
wherein said central working lumen is capable of acting as a conduit for the delivery therethrough of:
at least one additional secondary catheter,
at least one wire separate from said primary catheter, and
at least two pulley wires,
wherein said at least two pulley wires are located axially substantially within the wall of said single primary catheter, and capable of actively steering said at least two steerable segments; and
at least one pulley steering system for each of said at least two pulley wires;
wherein said at least two steerable segments each comprise said at least one pulley steering wire system for each segment, each of said at least one pulley steering wire system capable of steering each of said at least two steerable segments in a single direction of pulley wire pull,
said at least two steerable segments are composed of a primary steering segment and a secondary steering segment;
wherein said primary steering segment is:
located along any desired portion of said primary catheter within 10 cm of said distal end hole;
at least 0.1 cm long and no longer than 5 cm long; and
capable of being actively curved/bent from zero degrees to 180 degrees
wherein said secondary steering segment is:
located along any secondary segment located along a segment of said primary catheter, proximal to said primary segment, between 2 cm and 30 cm from said distal end hole;
at least 0.4 cm long and no more than 15 cm long,
capable of steering and creating a bend/curve, via a pulley effect on said pully wire, of zero degrees to 270 degrees; and
disposed on the same side, and curving in substantially the same initial fashion, as the primary curve/bend;
an external termination device which is in communication with said proximal end hole;
wherein said primary catheter further comprising at least one balloon affixed to the outer surface and proximal to said distal end hole; and at least one additional lumen substantially within the wall of the effective segment of said primary catheter which functions to inflate and deflate said at least one balloon; and
wherein said at least one balloon is capable of inflating and thereby are capable of:
altering flow in targeted vessels, and
anchoring said device; and
at least two treatment devices.

41. The device of claim 8, further comprising at least two treatment devices.

42. The device of claim 40, wherein said at least two treatment devices comprise at least one angioplasty balloon and at least one stent.

43. The device of claim 42, wherein said stent is mounted on said angioplasty balloon.

44. The device of claim 42, wherein said stent is self-expanding.

45. The device of claim 41, wherein said at least two treatment devices comprise at least one angioplasty balloon and at least one stent.

46. The device of claim 45, wherein said stent is mounted on said angioplasty balloon.

47. The device of claim 45, wherein said stent is self-expanding.

48. A method of using the device of claim 1 for establishing trans-radial arterial access to a multitude of end vessels, via a single site of radial access, said method comprising the steps of:
inserting an access needle through a percutaneous tissue tract leading to a radial artery
inserting a wire through said needle, into said radial artery
removing said needle
inserting the device of claim 1 over said wire
removing said wire
advancing said device of claim 1 over another wire, under fluoroscopic guidance, near a target vessel
removing said wire
steering and moving said device of claim 1 into target location
optionally repositioning the distal end hole of device of claim 1 repeatedly to access sequentially multiple successive target vessels, without removing the proximal portion of said device of claim 1 from said radial artery access site.

49. The method of claim 48, with further step of positioning at least one segment of said device abuts a vascular arch, and subsequently curves away from said vascular arch, so that it effectively utilizes said vascular arch as a fulcrum, to prevent kickback and prolapse of said device as well as of any additional devices passed therethrough

50. The method of claim 49, wherein the distal end of said device is further repositioned sequentially into multiple target vessels via a single initial radial access, wherein said target vessels include the right vertebral artery, the right internal carotid artery, the left internal carotid artery, and the left vertebral artery, in any order.
